(19) United States
(12) Patent Application Publication
(10) Pub. No.: US 2011/0125022 A1
(43) Pub. Date: May 26, 2011

(54) SYNCHRONIZATION FOR MULTI-DIRECTIONAL ULTRASOUND SCANNING

(75) Inventor: Roee Lazebnik, San Jose, CA (US)

(73) Assignee: SIEMENS MEDICAL SOLUTIONS USA, INC., Malvern, PA (US)

(21) Appl. No.: 12/625,888

(22) Filed: Nov. 25, 2009

(51) Int. Cl. A61B 8/14

(52) U.S. Cl. 600/444

(57) ABSTRACT

Multi-directional ultrasound scanning is synchronized. A plurality of wobbler arrays are used sequentially. To limit artifacts caused by motion, the sequential operation is synchronized. While a first wobbler array is scanning, a second wobbler array is moving or active. Once the first wobbler array completes a scan or portion of the scan, the second wobbler array begins the scan without waiting for initiation of the wobbling. The position of the second array may alternatively or additionally be synchronized with the first array or the end of the scan of the first array. The data from the different scans may represent overlapping volumes, so may be combined to form an extended field of view.

### BACKGROUND

The present embodiments relate to ultrasound scanning. In particular, the embodiments relate to scanning for different directions.

A conventional ultrasound exam is performed using a single handheld transducer. The transducer acquires plane information within a field of view (FOV) limited by the transducer design. There are many clinical applications, including fetal imaging, for which this approach prevents visualization of the entire anatomy of interest. Instead, multiple independent views are typically required to completely visualize the anatomy of interest. The sonographer moves the handheld transducer to different positions and independently acquires data at each position. Separate images are generated from the data acquired at each position.

Information for a volume may be acquired with a handheld transducer. For example, a wobbler transducer mechanically moves an array for electronic scanning in different planes. However, the FOV is also limited by the transducer design, so the entire anatomy of interest may not be viewed. The transducer may be positioned at other locations for scanning other regions, but motion of the fetus or within the region may result in difficult comparison of the images from different scans.

### BRIEF SUMMARY

By way of introduction, the preferred embodiments described below include a method, system, instructions, and computer readable media for synchronizing multi-directional ultrasound scanning. A plurality of wobbler arrays are used sequentially. To limit artifacts caused by motion, the sequential operation is synchronized. While a first wobbler array is scanning, a second wobbler array is moving or active. Once the first wobbler array completes a scan or portion of the scan, the second wobbler array begins the scan without waiting for initiation of the wobbling. The position of the second array may alternatively or additionally be synchronized with the first array or the end of the scan of the first array. The data from the different scans may represent overlapping volumes, so may be combined to form an extended field of view.

In a first aspect, a system is provided for synchronizing multi-directional ultrasound scanning. At least first and second wobbler transducers connect with a frame. The frame is configured to allow for independent movement of the first wobbler transducer relative to the second wobbler transducer. The independent movement is in translation along at least a first dimension, rotation about at least a second dimension, or combinations thereof, where the first and second dimensions are different or the same. An ultrasound imaging system is configured to sequentially scan an internal region of a patient with the first wobbler transducer and then with the second wobbler transducer. The sequential scans having overlapping fields of view such that a first volume scanned by the first wobbler transducer overlaps with a second volume scanned by the second wobbler transducer. The ultrasound imaging system is configured to generate an image as a function of data from the scan with the first wobbler transducer, data from the scan with the second wobbler transducer, and a relative position of the first and second volumes. A processor is configured to synchronize an array of the second wobbler transducer with the scan of the first wobbler transducer such that the second wobbler is ready to scan when the scanning shifts from the first wobbler transducer to the second wobbler transducer. A display is operable to display the image.

In a second aspect, a method for synchronizing multi-directional ultrasound scanning is provided. A patient is acoustically scanned with a first mechanically moved array. The scanning is of at least a first field of view of the first mechanically moved array. A second mechanically moved array is operated in an active mode without acoustic scanning during the acoustic scanning with the first mechanically moved array. The acoustic scanning with the first mechanically moved array is ceased. The patient is acoustically scanned with the second mechanically moved array after the ceasing and while still in the active mode. The scanning with the second mechanically moved array is of at least a second field of view of the second mechanically moved array where the second field of view different than but overlapping with the first field of view. Data from the scanning with the first mechanically moved array and from the scanning with the second mechanically moved array is combined as a function of a relative position of the first and second mechanically moved arrays. An image is generated as a function of the combining.

In a third aspect, a computer readable storage medium has stored therein data representing instructions executable by a programmed processor for synchronizing multi-directional ultrasound scanning. The storage medium includes instructions for sequentially scanning with two different transducer arrays; synchronizing movement of a first of the two different transducer arrays with an end of scan time of a second of the two different transducer arrays; and generating an image as a function of data from the sequential scanning with the two different transducer arrays.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of an ultrasound system for synchronizing multi-directional ultrasound scanning;

FIG. 2 is a graphical representation of an example frame for holding the transducers of the ultrasound system of FIG. 1;

FIG. 3 is a flow chart diagram of one embodiment of a method for synchronizing multi-directional ultrasound scanning.

### DETAILED DESCRIPTION OF THE DRAWINGS AND PRESENTLY PREFERRED EMBODIMENTS

Synchronization of two or more mechanical wobbler transducers may allow for more rapid acquisition. A large FOV may be composited using spatial encoding information from each transducer. Multiple transducers with overlapping fields of view are used for compounding a volume or planes representing an expanded field of view.

The compounded information may be used for quantification and/or imaging. For example, obstetrical imaging is provided. Whole fetus scanning may be provided. Sonographic visualization of other large anatomical structures may be provided using an array of transducers. The array of transducers is composed of independently positioned transducers with overlapping fields of view (FOV). Each transducer may be addressed serially or simultaneously throughout the array of transducers such that a composite large FOV volume may be assembled. Composition of the resulting volume is performed using knowledge of the individual transducer's geometry and orientation and/or using image processing techniques.

An array of transducers scanning overlapping regions may be used to reduce speckle. Although a given subvolume within the composite volume may be included in several individual transducers' FOV, each transducer may interrogate this subvolume from a different orientation. The speckle pattern as well as attenuation associated with the interrogating beam may differ between transducers. By compounding the information for a given subvolume from several transducers, both contrast and spatial resolution may be improved.

The different scan directions used by the transducers may reduce shadow artifacts. Shadows are created where a deep structure is obscured due to a reflective superficial or more shallow structure. A given transducer may not adequately visualize a subvolume within the transducer's field of view. Another transducer at a different orientation may visualize the same structure more effectively.

By synchronizing the scans between the transducers, multiple views may be acquired despite motion. Motion may result in artifacts or difficulty aligning data from different scans (e.g., transducers at different orientations or locations). High quality registration of the subvolumes may be difficult without accurate spatial information as to each transducer's location and orientation. The relative spatial position is determined using sensors on the transducers, sensors on a positioning device (e.g., robot), and/or correlation of data. Data correlation to determine relative position may be difficult where the motion alters the tissue being scanned. Synchronization may reduce time between sequential scans, resulting in less motion artifact.

The different acoustic paths traveled by each transducer's beam may result in varying levels of attenuation, phase aberration, and other image affective parameters. To account for this variation, each transducer's contribution to overlapping regions of the composite volume may be weighted.

The transducers may be physically large and heavy. A robot, support arm, belt, or other device may assist the user in positioning or holding the transducers.

Driving multiple volumetric transducers serially or simultaneously is performed with an ultrasound imaging system. To avoid frequency separation or other coding to distinguish scans from multiple arrays at a same time, sequential scanning may be used. Alternatively, frequency separation or other coding distinguishes the transmissions.

FIG. 1 shows a system 10 for synchronizing multi-directional ultrasound scanning. The system 10 includes two or more transducers 12, 16, location devices 14, an ultrasound imaging system 18, a processor 20, a memory 22, and a display 24. Additional, different, or fewer components may be provided. For example, the system 10 does not include the location devices 14. As another example, the system 10 includes a user interface. In one embodiment, the system 10 is a medical diagnostic ultrasound imaging system. In other embodiments, the processor 20 and/or memory 22 are part of a workstation or computer different or separate from the ultrasound imaging system 18. The workstation is adjacent to or remote from the ultrasound imaging system 18.

The transducers 12, 16 are single element transducers, linear arrays, curved linear arrays, phased arrays, 1.5 dimensional arrays, two-dimensional arrays, radial arrays, annular arrays, multidimensional arrays, or other now known or later developed arrays of elements. The elements are piezoelectric or capacitive materials or structures. The transducer 12 is adapted for use external to the patient, such as including a hand held housing or a housing for mounting to an external structure. Two transducers 12, 16 are shown, but three, four, or more transducers 12, 16 may be provided. Different ones of the transducers 12, 16 may have the same or different structure, such as one transducer being a linear array and another being a curved linear array. The transducers may be configured to scan an identical or different sized FOV. Each transducer's imaging parameters (frequency, depth, and others) may also be identical or different from other transducers.

In one embodiment, one or more, such as all, of the transducers 12, 16 are wobbler arrays. The wobbler arrays each include an array of transducer elements. The array of elements may be used to scan a region, such as electronic scanning of a plane. Belts, gears, pulleys, cams, and/or other devices connect with the array. A motor, such as an electric motor, drives the devices to move the array. The array is translated along a plane or curved plane and/or rotated. Due to motor operation and/or the device, the array may be moved back and forth between two limits, wobbling the array, within the probe housing. The limits may be mechanically or electrically determined.

Each transducer 12, 16 converts between electrical signals and acoustic energy for scanning a region of the patient body. The region of the body scanned is a function of the type of transducer array and position of the transducer 12 relative to the patient. For example, a linear transducer array in a wobbler may scan a plurality of rectangular or square, planar regions of the body. As another example, a curved linear array in a wobbler may scan a plurality of pie shaped regions of the body. Scans conforming to other geometrical regions or shapes within the body may be used, such as Vector® scans.

The planes are spaced apart due to movement of the array. The planes represent a volume of the patient. Different planes may be scanned by moving the array, such as by rotation, rocking, and/or translation. Alternatively, a volume is scanned by electronic steering alone (e.g., volume scan with a two-dimensional array).

The wobblers may include respective sensors configured to determine array positions, providing corresponding scan plane positions. The position of each planar scan is measured or known. For example, an encoder or other sensor determines the position of the array within its range of motion to determine the position of a given scan plane. Alternatively, the current draw of the motor or other feedback is provided to determine the position. Data de-correlation or other techniques may be used to determine the positions of scan planes acquired with a same array. In another alternative, the acquisition of each scan plane is triggered. The planes are acquired at set relative positions. In other embodiments, the array or motor speed over the range of motion may be known or determined. The speed profile, the number of scans, and the scan timing may be used to determine a position of each scan.

Optionally, the transducers 12, 16 include a location device 14. The location device 14 is in or on the ultrasound transducer 12, 16. For example, the location device 14 is mounted on, placed within, or formed as part of the housing of the transducer 12, 16. Signals or data are provided from or to the location device 14 with wires in the transducer cable or wirelessly.

The location device 14 is a sensor or sensed object. For example, the location device 14 includes coils of a magnetic position sensor. Three orthogonal coils are provided. By sequencing transmission through remote transmitter coils and measuring signals on each of the sensors coils, the location and orientation of the sensor coil is determined. The coils sense a magnetic field generated by another device external to the sensor. Alternatively, the magnetic field is generated by the location device 14, and coils spaced from the location device 14 sense the position information of the transmitter.

The location device 14 determines the location of the probe or transducer 12, 16, such as relative to a room space or other transducers 12, 16. The location device 14 indicates the relative positions of scanned volumes or planes acquired with different transducers 12, 16.

Other location devices 14 may be used. For example, a gravity sensor indicates the orientation of the transducer relative to the center of the earth. In other examples, the location device 14 is an accelerometer or gyroscope. An optical sensor may be used, such as the location device 14 being a pattern, light transmitter, or the housing of the transducer 12, 16. A camera images the transducer 12. A processor determines the orientation and/or position based on the location in the field of view, distortion, and/or size of the location device 14.

Other orientation sensors may be used for sensing one, two or three degrees of orientation relative to a reference. Other position sensors may be used with one, two or three degrees of position sensing. In other embodiments, a position and orientation sensor provide up to 6-degrees of position and orientation information. Examples of magnetic position sensors that offer the 6 degrees of position information are the Ascension Flock of Birds and the Biosense Webster position-sensing catheters.

In another embodiment, the location device 14 is a fiber optic position sensor, such as the Shapetape sensor available from Measurand, Inc. The orientation and/or position of one end or portion of the fiber optic position sensor relative to another end or portion are determined by measuring light in fiber optic strands. One end or other portion of the fiber optic position sensor is held adjacent to a known location. The bending, twisting, and rotation of the fiber optic positions sensor is measured, such as measuring at a time after the transducer is positioned adjacent an acoustic window. The relative position of the transducer at different acoustic windows may be determined.

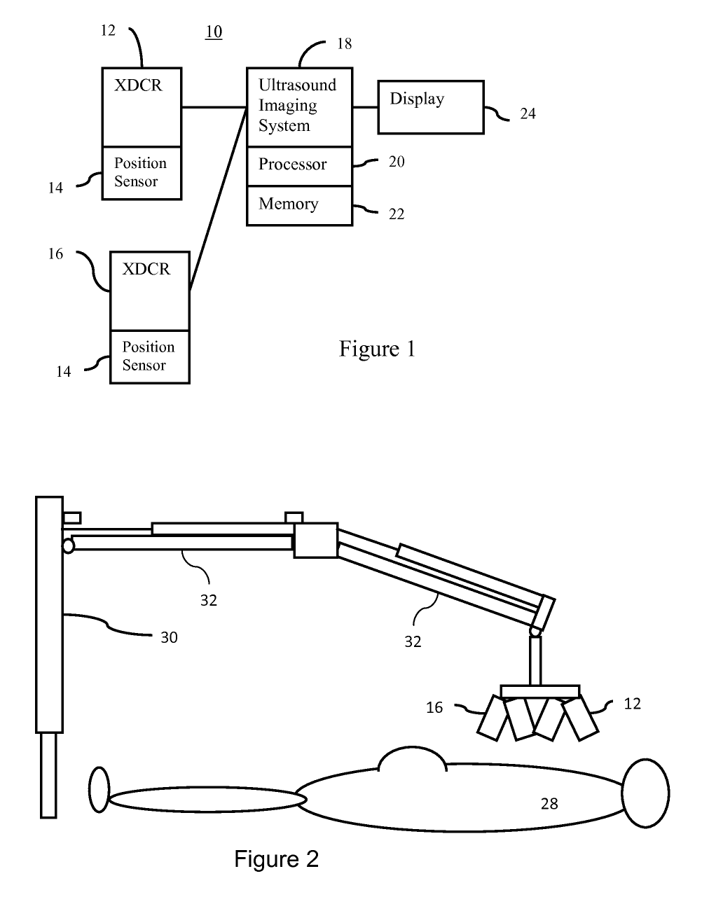

To assist the user in positioning and/or holding the transducers 12, 16, a frame 30 may be provided as shown in FIG. 2. The frame 30 is a pulley, belt, or other device for actively or passively reducing the weight required by the user to hold the transducers 12, 16. In one embodiment, the frame 30 includes shocks, motors, limiters, pumps, or other devices. The frame 30 may resist movement, lock, unlock, or ease movement.

In one embodiment, the frame 30 includes one or more support arms 32. The support arms 32 have any shape and size, such as being metal or plastic tubes, beams, or plates. The support arms 32 directly or indirectly connect with the transducers 12, 16. In one example embodiment, the support arm 32 is part of a robot or robotic assist system, such as the ACUSON 52000 Automated Breast Volume Scanner by Siemens Medical Solutions USA, Inc. The transducers 12, 16 are mounted on a same supporting arm or different supporting arms 32 such that a human operator does not need to hold any part of the transducers 12, 16 during imaging. The supporting arms 32 may be articulated, expandable, compressible, bendable, rotatable or otherwise moveable so as to support a wide variety of transducer positions relative to the patient 28. In the embodiment shown in FIG. 2, the support arms 32 are supported by a lift or moveable a column. Ceiling, floor, or wall mounts may be used. Tracked, fixed, rotatable, or other mounts may be used. In the example of FIG. 2, four mechanical wobbler transducers 12, 16 suitable for transabdominal fetal scanning of the patient 28 are shown.

The frame 30 is configured to allow for independent movement of the wobbler transducers 12, 16 relative to each other. The mechanical linkage allows for at least one transducer 12, 16 to move relative to another of the transducers 12, 16. The independence may be provided in one, two, or three degrees of translation and/or rotation. For example, one transducer 12 may be moveable to rotate with or without limits about two axes without also requiring rotation of another one of the transducers 16. The different transducers 12, 16 may be translatable and/or rotatable about the same or different dimensions.

The independence of motion may be provided by having at least one separate connection to the support arm 32. For example, each transducer 12, 16 connects with the frame 30 and/or support arm 32 with a separate joint or arm. Different groups of transducers 12, 16 may connect with a common support arm 32 different than a support arm 32 for another group of the transducers 12, 16. In one embodiment, four or other number of transducers 12, 16 connect with a common plate or other support arm 32. The relative position of the connections space the transducers 12, 16 for ease of positioning on the patient 28, such as for positioning around an abdomen of a pregnant patient.

Each transducer 12, 16 may be manipulated manually or automatically such that the relative position to each other is customizable. A handle and/housing is used by a user to manually move the transducer 12, 16. The support arm 32, connection, joint, or frame 30 may resist, assist, or freely allow the manual positioning. For example, the transducer 12, 16 may be locked and unlocked relative to the support arm 32 such that free motion is allowed when unlocked and motion is prevented unless over a certain amount of force is applied in a locked state. Automatic movement may be provided by motors or pumps with the guidance of the user and/or based on sensor feedback.

The spatial location and/or orientation of each transducer 12, 16 are determined using the location devices 14, such as robotic positioning sensors or sensors to detect translation and/or rotation in the allowed directions. The relative position, absolute position, and/or change in position may be used. As an alternative or in addition, the scan data is correlated to determine relative position. For determining the spatial location and/or orientation, any limits on motion of the transducers 12, 16 relative to each other may be used.

The support arms 32 are moveable to position the transducers 12, 16 adjacent to the patient 28. For example, a resistance device, motor, or both are used to position the transducers 12, 16 adjacent an abdomen of the patient 28. The support arms 32 are then locked or maintained in position. For example, a shock or other resistance device may counter a portion of the force caused by gravity and motion in other directions is locked. If the transducers 12, 16 need to be moved away, the support arms 32 are lifted against the remaining force of gravity. During scanning, the remaining gravity force maintains the transducers 12, 16 against the patient. Once the support arms 32 are positioned to locate the transducers 12, 16 by the desired region of the patient, the transducers 12, 16 may be moved to the desired acoustic windows.

Referring to FIG. 1, the ultrasound imaging system 18 is a medical diagnostic ultrasound system. For example, the ultrasound imaging system 18 includes a transmit beamformer, a receive beamformer, a detector (e.g., B-mode and/or Doppler), a scan converter, and the display 24 or a different display. The ultrasound imaging system 18 connects with the transducers 12, 16, such as through one or more releasable connectors. Transmit signals are generated and provided to a selected transducer 12, 16. A multiplexer or connector receptacle selection selects the transducer 12, 16 to be used for scanning at any given time. Responsive electrical signals are received from the selected transducer 12, 16 and processed by the ultrasound imaging system 18. The ultrasound imaging system 18 causes a scan of an internal region of a patient with the transducer 12, 16 and generates data representing the region as a function of the scanning. The data is beamformer channel data, beamformed data, detected data, scan converted data, and/or image data. The data represents anatomy of the region, such as the heart, liver, fetus, muscle, tissue, fluid, or other anatomy.

In another embodiment, the ultrasound imaging system 18 is a workstation or computer for processing ultrasound data. Ultrasound data is acquired using an imaging system connected with the transducer 12 or using an integrated transducer 12 and imaging system. The data at any level of processing (e.g., radio frequency data (e.g., I/Q data), beamformed data, detected data, and/or scan converted data) is output or stored. For example, the data is output to a data archival system or output on a network to an adjacent or remote workstation. The ultrasound imaging system 18 processes the data further for analysis, diagnosis, and/or display.

Using a multiplexer or other structure and programming, the imaging system 18 is configured to sequentially scan an internal region of the patient with the different transducers 12, 16. Signals are transmitted to and received from one of the transducers 12, 16 at a given time. For example, one transducer 12 is used to scan a volume. Another transducer 16 is then used to scan another volume. The transmit and receive signals are beamformed as appropriate for scanning with the type of transducer 12, 16. Alternatively, more than one transducer 12, 16 may be selected and scan at a same time.

These sequential scans have overlapping fields of view. The transducers 12, 16 are positioned and the scan format selected to cause the field of view of the transducers 12, 16 to at least partially overlap. A volume scanned by one transducer 12 overlaps with a volume scanned by another transducer 16. The transducers 12, 16 are addressed serially or in an arbitrary order by the imaging system 18 such that one or several of the transducers 12, 16 are imaging at a given time. For example, in the case of four mechanical wobbler transducers 12, 16, all of the transducers 12, 16 may be wobbling internally throughout their sweep configuration but only one transducer at a time is utilized for imaging. Alternatively, non-overlapping fields of view and/or simultaneous scanning are used.

The imaging system 18 generates an image from the scan data. Beamformation, detection, scan conversion, and/or rendering are used to generate each image. Separate images may be generated for the data from separate transducers 12, 16. The data may be combined, such as combining pre or post detection into a set of data representing a scan volume, a sub-volume, a plane, an extended field of view plane or an extended field of view volume. Extended field of view is a field of view greater than obtainable with a complete scan using a single transducer 12, 16 at one position.

In one embodiment, the image is generated as a rendering of data representing a three-dimensional region. A data set is formed by combining data from two or more transducers. The data set represents only the overlapping portions or an extended field of view. Once volume data is independently acquired by all participating transducers 12, 16, a composite volume is assembled.

The scan volumes are spatially aligned (registered). In one embodiment, the location devices 14 are used for aligning the regions represented by the data. The location devices 14 indicate positions of the transducers 12, 16 during respective scans. Absolute or relative position information may be used.

For data-based registration, cross-correlation, minimum sum of absolute differences, or other similarity function is used to identify the relative translation and/or orientation of the regions. The best or sufficient match of the data to each other is determined. The translation and/or rotation associated with the match indicate the different or relative positions of the regions represented by the data. The match spatially aligns the data from the scans for the different fontanels.

Multiple sources of alignment information may be used. For example, both data-based and sensor-based relative positions and orientations are determined. Average position and orientation are used. One source may be used for position and another source may be used for orientation. One source may be used to assure that a primary source is correct.

In one embodiment, initial relative position estimates are provided by the location device 14 associated with each transducer 12, 16. Additional accuracy may be obtained through data correlation. The initial position is used to limit the search space, provide an initial location for searching, or more quickly determine a strongest correlation. The data sets are translated and/or rotated relative to each other in order to identify a relative position with a greatest similarity.

Once aligned, the data is combined. The data from different scans are compounded as a function of the spatial alignment. Where data from multiple sets or different scans represents a same spatial location, the data is combined, such as averaged. Due to the different scan formats and/or different acoustic windows, the data may generally represent a same spatial location, but not exactly align. Data from one or more scans may be converted or formatted to a grid associated with another of the scans or a reference grid. For example, the data representing different volumes is interpolated to a three-dimensional reference grid. After conversion, values for data from multiple volumes are combined. Alternatively, a nearest neighbor, interpolation, or other approach is used to determine the data to be combined.

Since the scanned volumes may not be identical, different spatial locations may be associated with a different number of values to be combined. For example, one spatial location may be represented by a single value from one scan. Another spatial location may be represented by two values from scans by two transducers 12, 16. Another spatial location may be represented by three values, one from each of three transducers 12, 16. Normalized or averaged combination is used. Filtering may be provided to reduce any artifacts from combining different numbers of values for different spatial locations.

The values are combined by averaging. Other combination functions may be used, such as a maximum or minimum value selection. In one embodiment, a weighted average is used. The values are weighted prior to averaging. The weighting may be predetermined or fixed. For a simple average, the weights are set based on the number of contributing values.

In one embodiment, the weights adapt as a function of the spatial location, data quality, or combinations thereof. For example, near field or mid field information may be better quality than far field or very near field data. Data in the middle of a scan field may be better quality than data associated with larger steering angles. The better quality data is weighted more heavily. For example, near field data is weighted more heavily than far field data. Wobbler transducers may provide better quality information for one array orientation than another, such as due to speed of movement of the array. The better quality data may be weighted more heavily.

The data may be processed to determine the quality or a quality factor. For example, the noise level associated with different spatial locations is determined. The standard deviation in a generally homogenous region may indicate a level of noise for the scan or a portion of the scan. As another example, a measure of high frequency variation indicates the noise level. In another example, the magnitude of the return without time or depth gain compensation is compared to a threshold level or slope to determine a noise level as a function of depth. Noise levels may be determined for different portions of a scan. The noise at other locations in interpolated. The quality for a given value is indicated by the level of noise.

Any variance or difference in weighting may be used. The weighting is relative, such as all the weights adding to unity. A difference in quality between values may be determined and the relative weighting set based on the difference. For example, if two values have similar quality, then equal weighting is provided. If the two values have different quality, then unequal weighting is provided. One or more factors may be used to determine overall quality. The factors may be weighted differently depending on importance or reliability.

The relative weights for the contributing scans may be selected based on echogenicity. Stronger weighting is provided for higher intensity values. Other considerations may be used to adapt the weights. The registration may be used for weighting. Better correlation may indicate more equal weighting is appropriate. Poor correlation may indicate stronger weighting for one or more data sets, such as the data closest to the respective array. Given two contributing data values for a given location, the data value from a scan by a closer array is more heavily weighted.

The display 24 is a CRT, LCD, projector, plasma, printer, or other display for displaying two-dimensional images or three-dimensional representations. The display 20 displays ultrasound images as a function of the output image data. For example, a multi-planar reconstruction (MPR) of two or more images representing orthogonal planes is provided. As another example, a plurality of ultrasound images representing two or more parallel planes in the internal region are provided. Volume or surface rendering may alternatively or additionally be used.

The composite volume is used for quantification, imaging, and/or archiving. The data of the composite volume may be segmented or border detection applied to determine volume values or isolate information associated with particular structures. The dataset representing the composite volume may be output as image data. The image data may be data at any stage of processing, such as prior to or after detection. The image data may be specifically formatted for display, such as red, green, blue (RGB) data. The image data may be prior to or after any mapping, such as gray scale or color mapping.

The processor 20 is one or more general processors, digital signal processors, application specific integrated circuits, field programmable gate arrays, controllers, analog circuits, digital circuits, server, combinations thereof, network, or other logic devices for controlling the transducers 12, 16 and/or corresponding scans. A single device is used, but parallel or sequential distributed processing may be used. In one embodiment, the processor 20 is a system controller of the ultrasound imaging system 18. The processor 20 receives inputs from any location device 14, the transducers 12, 16, and/or the ultrasound imaging system 18.

The processor 20 synchronizes the array of one or more wobbler transducers 12, 16 with the scan of another wobbler transducer 12, 16. While a first transducer 12 is scanning, one or more other transducers 16 are synchronized to reduce transition between scans. The other transducers 16 are synchronized to the same or different transducer 12, 16. The other transducers 16 are synchronized such that the transducer 16 is ready to scan when the scanning shifts from the currently scanning transducer 12 to the waiting transducer 16. The waiting transducer 16 is synchronized to the currently scanning transducer 12, an array position of the currently scanning transducer 12, an end time of the scan by the currently scanning transducer 12, an end scan plane position of the currently scanning transducer 12, or other aspect of the current scan or transducer 12.

The waiting transducers 16 are synchronized for optimal acquisition speed or to increase acquisition speed. For example, while a first transducer 12 is imaging (active mode), three other transducers 16 are in standby mode. When the first transducer 12 completes an imaging sweep through its FOV, the first transducer 12 is placed into standby mode and a second transducer 16 becomes active and begins imaging immediately or with little delay. The synchronization provides the array of the subsequent transducer 16 in a desired location, at a desired rate of movement, or at a desired level of activity. For example, the synchronization provides the array at an origin position relative to a range of sweep. Each of the transducers is addressed serially such that imaging information is only obtained from a single transducer at a given time, but the transducers are in standby mode to allow for reduced transition time. A large field of view with motion may be scanned but with fewer artifacts.

The synchronization is provided by control of the transducer 12, 16. For example, the wobbler is switched on. The array of the second wobbler transducer 16 is synchronized with the scan of the first wobbler transducer 12 by activating the second wobbler transducer 16 prior to the scanning shift from the first wobbler transducer 12 to the second wobbler transducer 16. At a given time, each transducer 12, 16 is in active, standby, or deactivated mode. Active mode is where the transducer 12 is imaging or scanning such that acoustic content is transmitted and/or received by the transducer 12 and communicated to the imaging system 18 in real-time. Standby mode is utilized while the transducer 16 is not transmitting or receiving acoustic content by is ready to do so instantly or with little delay. In the case of a mechanical wobbler transducer 12, 16, the array is already up to speed or is moving (“wobbling”) but not transmitting acoustic pulses. The time to come up to speed is reduced or eliminated by the synchronization.

As another example, the synchronization is based on array position. A waiting array of a wobbler is positioned to be at a particular location in a sweep at the scanning shift from another transducer 12 to the waiting transducer 16. For example, the movement of the array is timed to be at a limit or center of a sweep of motion or wobble at the time of beginning to scan. Rather than waiting for the array to move to the desired location after a scan with another array is complete, the waiting array is timed to be close or at the location at the transition. Any desired array location may be used, such as a location corresponding to the end of a previous scan. The positioning may be achieved by controlling the speed of motion of the array and/or the start time of moving the array.

One or more transducers 12, 16 may not be used for a given situation. These transducers 12, 16 may be deactivated or in standby but not used. To avoid noise or undesired oscillation, a deactivated mode is used where the array. The electrical components (e.g., motor) of the deactivated transducer are inactive or not energized. Alternatively, the deactivated mode may be used where one or more transducers 12, 16 are to be used in the scan sequence before the current transducer 12, 16. Once the time of use for the current transducer 12, 16 is closer, then the transducer 12, 16 is moved from the deactivated mode to a standby mode as part of the synchronization.

The memory 22 is a tape, magnetic, optical, hard drive, RAM, buffer or other memory. The memory 22 stores the data from the different scans and/or the data of the composite volume.

The memory 14 is additionally or alternatively a computer readable storage medium with processing instructions. Data representing instructions executable by the programmed processor 20 and/or the imaging system 18 is provided for synchronizing multi-directional ultrasound scanning. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

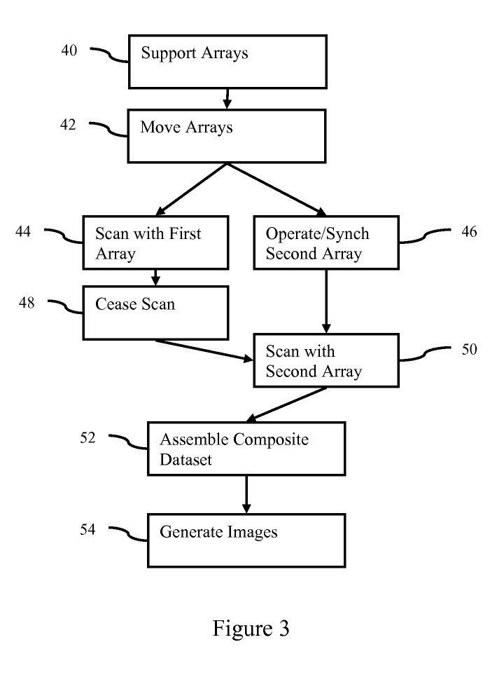

FIG. 3 shows a method for synchronizing multi-directional ultrasound scanning. The acts of FIG. 3 are implemented by the system 10 of FIG. 1 or a different system. The acts are implemented with the assistance of the frame 30 of FIG. 2 or without. The acts are performed in the order shown or a different order. Additional, different, or fewer acts may be performed. For example, acts 40, 42, 52, and/or 54 may not be used. As another example, additional synchronizing acts 48 are provided for other transducers. Each transducer sequentially performs acts 44 and 46 while other transducers are performing act 48.

In act 40, two or more arrays are supported. The support is a belt, robot, or other support structure. The support connects, directly or indirectly, the two probes for the arrays together. The support may be moved to move all of arrays. For example, a support structure is used by a sonographer. The arrays are positioned together by the user near a patient. The user applies force to the array probes and/or the support structure. The user positions the support structure. The arrays are positioned adjacent the patient, such as over or against an abdomen of the patient. During positioning, the support structure generally maintains equilibrium with gravity. The user applies force to overcome this equilibrium or other friction. In alternative embodiments, force applied by motors or other sources than the user positions the support structure.

The ultrasound transducer support structure may be locked. Brakes are applied, such as mechanical limiters positioned to prevent motion. The user activates a switch. In response, a controller causes the brakes to activate. For example, servo or stepper motors position brake pads against a surface, engage gear locks, freeze joint motors, adjust pins, or perform another action to lock the frame 30. Alternatively, the user manually locks one or more brakes. In other embodiments, locking is not provided. Instead, equilibrium is used. The resistance to gravity or other motion holds the support structure sufficiently in place.

In one embodiment, two or more different arrays are separately supported from a common support arm. Separate connections of the probe housings with the common support arm are provided. The common support arm is positioned adjacent to the patient such that the probe housings and corresponding arrays are adjacent to and/or against the patient.

In act 42, one or more of the arrays are further moved. The probe housing of the array is moved adjacent to the patient. For example, a joint or extension is unlocked. The probe is then translated and/or rotated to place an acoustic window for the array against the skin or gel on the skin of the patient. The joint or extension is then locked or left in position. The process is repeated for any probe housings to be used but not properly placed against the patient. Positioning the probe housing positions the array, at least in part, for scanning the patient.

The arrays are positioned independently of each other. The position of one probe may depend, in part, on the position of another probe. For example, the probes connect to a same frame or support arm, so are moveable together. The probes are independently moveable along at least one degree of freedom, at least within a range permitted by the connection. The probe and array are independent of other probes and arrays by being moveable separately or moveable while others are not being moved. Independent movement allows positioning of the arrays at the desired acoustic windows of patients of different sizes or shapes.

In one example, a pregnant patient is placed on a bed in the supine position. The arrays on the common support arm 32 are lowered such that one or more of the transducers 12, 16 is in contact with the patient's abdomen. Each transducer 12, 16 is independently positioned for optimal overlap and coverage of the maximum achievable composite fetal volume.

In act 44, one of the arrays is used for scanning. For a wobbler array, the array is started by mechanically oscillating the array. Transmit and receive signals are used to electronically steer for scanning from the moving array. Any type of scanning may be used, such as planar or volume scanning. For planar scanning, multiple planes are sequentially scanned. The transducer may be rocked, rotated, translated or otherwise moved to scan the different planes from the same acoustic window. For example, perpendicular planes are scanned by rotation of the transducer or aperture. Alternatively, a single plane is scanned.

The scanning may be for B-mode, color flow mode, tissue harmonic mode, contrast agent mode or other now known or later developed ultrasound imaging modes. Combinations of modes may be used, such as scanning for B-mode and Doppler mode data. Any ultrasound scan format may be used, such as a linear, sector, or Vector®. Using beamforming or other processes, data representing the scanned region is acquired.

The scanning is of a field of view. A patient is acoustically scanned to an extent provided by the array and/or as defined by the transmit and receive beamformation. The lateral (elevation and azimuth) and range is set by beamforming and limited by the size and shape of the array. For a wobbler transducer, the speed of the array mechanical movement and/or physical limit on the movement may limit the size of the volume scanned. The array scans at different positions along a sweep.

The patient is scanned sequentially with different transducer arrays. Each array scans a different volume. The volumes may or may not overlap. The scanning is from different acoustic windows. Any two or more different acoustic windows may be used.

While one array is scanning in act 44, one or more other arrays are synchronized to the scanning array in act 46. The synchronization is provided by operation, movement of the array in the probe, array speed, array position, or other control of the waiting array based on the scan timing, operation, and/or position of the currently scanning array. Other operation may be used for synchronization. For example, bias voltages are applied to a waiting CMUT array.

In one embodiment, a waiting mechanically moved array is operated in a standby mode. The array is oscillated, rotated, translated or otherwise moved while the current array is scanning. For example, the waiting array is wobbled while waiting. The operation may occur during an entire time the current array is scanning or start any time before ceasing of the scan by the current array.

The waiting array is operated without acoustic scanning. For example, the next or other waiting arrays are wobbled while not scanning.

In another embodiment, movement is synchronized. The movement of the waiting array is synchronized with the current array or current scan. For example, movement of the waiting array is synchronized with an end of scan time of the current array. A starting position of the waiting array is identified. The starting position may be an end of sweep (e.g., furthest extent of translation or wobbling), center, or other position. The waiting array is operated so that the waiting array is at or approaching the starting position when the currently scanning array ceases scanning (i.e., at the end time of the previous scanning). The synchronization may be provided by increasing or decreasing a speed of the waiting array and/or by selection of the start time of movement of the waiting array prior to scanning with the waiting array.

In act 48, the currently scanning array completes the scan. Completion may be of a sub-region of the scan region of the current array. For example, the current array is capable of scanning 100 spaced apart planes. After scanning one or more, but not all, of the planes, the scanning is ceased until the current array's next turn. Frame or group of frames interleaving may be used between the different arrays due to the synchronization. Completion may be of one or more entire scans. For example, the current array scans all 100 planes once or more before ceasing.

After completion, the current array ceases scanning. The array stops being used for acoustic transmit and receive operation. The current array may continue to move, such as being synchronized to a waiting array or previously waiting and now scanning array. The scanning by all the arrays may cycle through each array multiple times, such as for real-time or on-going scanning. Alternatively, the current array is deactivated after ceasing the scanning. The current array may be used to scan again, such as being placed in standby when appropriate to synchronize to another array. The current array may not be used to scan again for a given image, imaging session, and/or patient.

In act 50, a waiting, synchronized array acoustically scans upon ceasing of scanning by the previous array. The waiting array is synchronized with the previous array or scan by the previous array, so the time between ceasing scan with the previous array and beginning acoustic scan with the waiting array is less than if the waiting array had to be started or brought up to speed. Since the waiting array is in the standby mode, the array is already moving, already at a desired speed, already at a desired position, close or approaching a desired position, or combinations thereof.

The scanning is performed as discussed above for act 44. The same or different scan format is used. Since a different array is used, the scan region or field of view is different. The scan region is a plane or volume. The scan region is entirely separate from or overlaps with the scan region of the previous and/or subsequent scanning array. For example, a volume scanned by a subsequent array overlaps with a volume scanned by a current and/or previous array. Each field of view may overlap with all other fields of view. Alternatively, one or more fields of view overlap with some but not all of the other fields of view.

Acts 46, 48, and 50 may repeated. The acts may be repeated where there are three or more arrays. Transitioning from a second array to a third array repeats the acts. The acts may be repeated where the scans are repeated by the same arrays. For example, the scan transitions from a second array back to a first array. The first array is synchronized with the scan or array position of the second array.

In act 52, data from different scans is combined. Data for the field of views from the different arrays is combined into a data set representing an extended field of view. The relative positions of the fields of view are determined by data correlation where the fields overlap. Where the scan does not overlap, the sensed positions of the different arrays are used. Both array position and data correlation may be used to align the data. The relative position of the fields of view is determined. The aligned data is combined by averaging, weighted averaging or other function. In alternative embodiments, data is not combined. Separate images are formed and combined. In other embodiments, no combination occurs. Separate images and/or quantification from separate data sets are used.

In act 54, an image is generated. The image is generated from the combined dataset. Alternatively, the image is generated as a combination of images created from different datasets. Data acquired from sequential scanning by different arrays is used to generate an image. For example, an extended field of view image is generated without intentional movement of the transducers. The extended field of view may extend over an entire region of interest beyond the ability of a single array, such as an entire fetus. In other embodiments, the image is not an extended field of view, but includes compounding from different look directions, reducing speckle and shadowing.

The image may be generated as a two-dimensional image from data representing a plane. An image from any arbitrary plane may be generated from the composite data representing a volume, such as a multi-planar reconstruction. Alternatively, one or more two-dimensional images are generated along a scan plane. The image may be generated as a rendering of a three-dimensional region. Surface or projection rendering may be used. The rendering is generated from data representing composited volumes, a sub-volume, an overlapping region, a single scan volume, or a plane.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A system for synchronizing multi-directional ultrasound scanning, the system comprising:
a frame;
at least first and second wobbler transducers connected with the frame, the frame configured to allow for independent movement of the first wobbler transducer relative to the second wobbler transducer, the independent movement being translation along at least a first dimension, rotation about at least a second dimension, or combinations thereof, the first and second dimensions being different or the same;
an ultrasound imaging system configured to sequentially scan an internal region of a patient with the first wobbler transducer and then with the second wobbler transducer, the sequential scans having overlapping fields of view such that a first volume scanned by the first wobbler transducer overlaps with a second volume scanned by the second wobbler transducer, the ultrasound imaging system configured to generate an image as a function of data from the scan with the first wobbler transducer, data from the scan with the second wobbler transducer, and a relative position of the first and second volumes;
a processor configured to synchronize an array of the second wobbler transducer with the scan of the first wobbler transducer such that the second wobbler is ready to scan when the scanning shifts from the first wobbler transducer to the second wobbler transducer; and
a display operable to display the image.

2. The system of claim 1 wherein the at least first and second wobbler transducers comprises the first, the second, third, and fourth wobbler transducers.

3. The system of claim 1 wherein the frame comprises a support arm connected with the first and second wobbler transducers, the first wobbler transducer having a separate connection to the support arm than the second wobbler transducer, the support arm having a resistance device, a motor, or both for maintaining the support arm at a position relative to the patient during the scanning.

4. The system of claim 1 wherein the first and second wobbler transducers include respective sensors configured to determine array positions, the processor configured to synchronize as a function of the array positions.

5. The system of claim 1 wherein the processor is configured to synchronize the array of the second wobbler transducer with the scan of the first wobbler transducer by activating the second wobbler transducer prior to the scanning shift from the first wobbler transducer to the second wobbler transducer.

6. The system of claim 1 wherein the processor is configured to synchronize the array of the second wobbler transducer with the scan of the first wobbler transducer by positioning the array at a particular location in a sweep of the array at the scanning shift from the first wobbler transducer to the second wobbler transducer.

7. The system of claim 6 wherein the particular location comprises a location at a limit of the sweep.

8. The system of claim 1 wherein the processor is configured to synchronize the array of the second wobbler transducer with the scan of the first wobbler transducer by increasing or decreasing a speed of the array.

9. The system of claim 1 wherein the at least first and second wobbler transducers comprises the first, the second, and third wobbler transducers, and wherein the processor is configured avoid activating the third wobbler transducer where the image is a function of the data from the first and second wobbler transducers and not data from the third wobbler transducer.

10. The system of claim 1 wherein the image comprises a rendering of a three-dimensional region including the first and second volumes.

11. A method for synchronizing multi-directional ultrasound scanning, the method comprising:
acoustically scanning a patient with a first mechanically moved array, the scanning being of at least a first field of view of the first mechanically moved array;
operating a second mechanically moved array in an active mode without acoustic scanning during the acoustic scanning with the first mechanically moved array;
ceasing the acoustic scanning with the first mechanically moved array;
acoustically scanning the patient with the second mechanically moved array after the ceasing and while still in the active mode, the scanning with the second mechanically moved array being of at least a second field of view of the second mechanically moved array, the second field of view different than but overlapping with the first field of view;
combining data from the scanning with the first mechanically moved array and from the scanning with the second mechanically moved array as a function of a relative position of the first and second mechanically moved arrays; and
generating an image as a function of the combining.

12. The method of claim 11 wherein operating comprises wobbling the second mechanically moved array while not scanning.

13. The method of claim 11 wherein operating comprises synchronizing a starting position of the second mechanically moved array with an end time of the scanning with the first mechanically moved array.

14. The method of claim 13 wherein synchronizing comprises operating the second mechanically moved array so that the second mechanically moved array is at a furthest extent of translation at the end time.

15. The method of claim 11 further comprising:
separately supporting the first and second mechanically moved arrays on a common support arm; and
moving the first mechanically moved array independently relative to the second mechanically moved array, the moving positioning the first and second mechanically moved arrays adjacent to the patient;
wherein the common support arm is configured to maintain the first and second mechanically moved arrays adjacent to the patient.

16. The method of claim 11 wherein the first and second field of views are first and second volumes, the first and second volumes overlapping, and wherein generating the image comprises rendering a three-dimensional region comprising the first and second volumes.

17. In a computer readable storage medium having stored therein data representing instructions executable by a programmed processor for synchronizing multi-directional ultrasound scanning, the storage medium comprising instructions for:
sequentially scanning with two different transducer arrays;
synchronizing movement of a first of the two different transducer arrays with an end of scan time of a second of the two different transducer arrays; and
generating an image as a function of data from the sequential scanning with the two different transducer arrays.

18. The computer readable storage medium of claim 17 wherein synchronizing movement comprises wobbling the first transducer array while not scanning.

19. The computer readable storage medium of claim 17 wherein synchronizing comprises synchronizing a starting position of the first transducer array with the end of the scan time of the second transducer array.

20. The computer readable storage medium of claim 19 wherein synchronizing comprises operating the first transducer array so that the first transducer array is at a furthest extent of translation at the end of the scan time of the second transducer array.

21. The computer readable storage medium of claim 17 wherein the instructions further comprise performing the synchronizing repetitively with frame or group of frame interleaving of the scanning with the two different transducer arrays.
